(19) United States
(12) Patent Application Publication
(10) Pub. No.: US 2022/0335615 A1
(43) Pub. Date: Oct. 20, 2022

(54) CALCULATING HEART PARAMETERS

(71) Applicant: FUJIFILM SONOSITE, INC., Bothell, WA (US)

(72) Inventors: Christopher Aleksandr White, North Vancouver (CA); Thomas Michael Duffy, Snohomish, WA (US); Davinder S. Dhatt, Woodinville, WA (US); Adam Benjamin Pely, Seattle, WA (US); William Andrew Needles, Toronto (CA)

(73) Assignee: FUJIFILM SONOSITE, INC., Bothell, WA (US)

(21) Appl. No.: 17/234,468

(22) Filed: Apr. 19, 2021

(51) Int. Cl. G06T 7/00; G06T 7/70; G06T 7/11; G06T 7/62; G06T 7/13; A61B 8/00; A61B 8/06

(52) U.S. Cl. CPC G06T 7/0016; G06T 7/70; G06T 7/11; G06T 7/62; G06T 7/13; A61B 8/463; A61B 8/469; A61B 8/065; G06T 2207/30048; G06T 2207/20081; G06T 2207/30168

(57) ABSTRACT

A method for calculating a heart parameter includes receiving a series of two-dimensional images of a heart, the series covering at least one heart cycle. The method includes calculating a volume of the heart in a first systole image based on an orientation of the heart in the first systole image and a segmentation of the heart in the first systole image, and a volume of the heart in a first diastole image based at least on an orientation of the heart in the first diastole image and a segmentation of the heart in the first diastole image; determining the heart parameter based at least on the volume of the heart in the first systole image and the volume of the heart in the first diastole image; determining a confidence score of the heart parameter; and displaying the heart parameter and the confidence score.

### FIELD OF DISCLOSED SUBJECT MATTER

The disclosed subject matter is directed to methods and systems for calculating heart parameters. Particularly, the methods and systems can calculate heart parameters, such as ejection fraction, from a series of two-dimensional images of a heart.

### BACKGROUND

Left ventricle (“LV”) analysis can play a crucial role in research aimed at alleviating human diseases. The metrics revealed by LV analysis can enable researchers to understand how experimental procedures are affecting the animals they are studying. LV analysis can provide critical information on one of the key functional cardiac parameters—ejection fraction—which measures how well the heart is pumping out blood and can be key in diagnosis and staging heart failure. LV analysis can also determine volume and cardiac output. Understanding these parameters can help researchers to produce valid, valuable study results.

Ejection fraction (“EF”) is a measure of how well the heart is pumping blood. The calculation is based on volume at diastole (when the heart is completely relaxed and the LV and right ventricle (“RV”) are filled with blood) and systole (when the heart contracts and blood is pumped from the LV and RV into the arteries). The equation for EF is shown below:

$$E F \% = 100 \times \frac{{V}_{dia} - {V}_{sys}}{{V}_{dia}} \qquad ( 1 )$$

Ejection fraction is often required for point-of-care procedures. Ejection fraction can be computed using a three-dimensional (“3D”) representation of the heart. However, computing ejection fraction based on 3D representations requires a 3D imaging system with cardiac gating (e.g., MRI, CT, 2D ultrasounds with 3D motor, or 3D array ultrasound transducer), which is not always available.

Accordingly, there is a need for methods and systems for calculating heart parameters, such as ejection fraction, for point-of-care procedures.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended figures. To achieve these and other advantages and in accordance with the purpose of the disclosed subject matter, as embodied and broadly described, the disclosed subject matter is directed to methods and systems for calculating heart parameters, such as ejection fraction using two-dimensional (“2D”) images of a heart, and for example, in real time. The ability to display heart parameters in real time can enable medical care providers to make a diagnosis more quickly and accurately during ultrasound interventions, without needing to stop and take measurements manually or to send images to specialists, such as radiologists.

In one example, a method for calculating a heart parameter includes receiving, by one or more computing devices, a series of two-dimensional images of a heart, the series covering at least one heart cycle, and identifying, by one or more computing devices, a first systole image from the series of images associated with systole of the heart and a first diastole image from the series of images associated with diastole of the heart. The method also includes calculating, by one or more computing devices, an orientation of the heart in the first systole image and an orientation of the heart in the first diastole image, and calculating, by one or more computing devices, a segmentation of the heart in the first systole image and a segmentation of the heart in the first diastole image. The method also includes calculating, by one or more computing devices, a volume of the heart in the first systole image based on the orientation of the heart in the first systole image and the segmentation of the heart in the first systole image, and a volume of the heart in the first diastole image based at least on the orientation of the heart in the first diastole image and the segmentation of the heart in the first diastole image. The method also includes determining, by one or more computing devices, the heart parameter based at least on the volume of the heart in the first systole image and the volume of the heart in the first diastole image, and determining, by one or more computing devices, a confidence score of the heart parameter. The method also includes displaying, by one or more computing devices, the heart parameter and the confidence score.

In accordance with the disclosed subject matter, the method can include determining areas for the heart including an area for the heart for each image in the series of images, wherein identifying the first systole image can be based on identifying a smallest area among the areas, the smallest area representing a smallest heart volume. The method can include determining areas for the heart including an area for the heart for each image in the series of images, wherein identifying the first systole image can be based on identifying a largest area among the areas, the largest area representing a largest heart volume.

Calculating the orientation of the heart in the first systole image and the orientation of the heart in the first diastole image can be based on a deep learning algorithm. The method can include identifying a base and an apex of the heart in each of the first systole image and the first diastole image, wherein calculating the orientation of the heart in the first systole image and the orientation of the heart in the first diastole image can be based on the base and the apex in the respective image. Calculating the segmentation of the heart in the first systole image and the segmentation of the heart in the first diastole image can be based on a deep learning algorithm. The method can include determining a border of the heart in each of the first systole image and the first diastole image, wherein calculating the segmentation of the heart in the first systole image and the segmentation of the heart in the first diastole image can be based on the orientation of the heart in the respective image and the border of the heart in the respective image.

The method can include generating a wall trace of the heart including a deformable spline connected by a plurality of nodes, and displaying the wall trace of the heart in one of the first systole image and the first diastole image. The method can include receiving a user adjustment of at least one node to modify the wall trace. The method can further include modifying the wall trace of the heart in the other of the first systole image and the first diastole image, based on the user adjustment. The heart parameters can include ejection fraction. Determining the heart parameter can be in real time. The method can include determining a quality metric of the images in the series of two-dimensional images, and confirming that the quality metric is above a threshold.

In accordance with the disclosed subject matter, a method for calculating heart parameters includes receiving, by one or more computing devices, a series of two-dimensional images of a heart, the series covering a plurality of heart cycles, and identifying, by one or more computing devices, a plurality of systole images from the series of images, each associated with systole of the heart and a plurality of diastole images from the series of images, each associated with diastole of the heart. The method also includes calculating, by the one or more computing devices, an orientation of each heart in each of the systole images and an orientation of the heart in each of the diastole images, and calculating, by one or more computing devices, a segmentation of the heart in each of the systole images and a segmentation of the heart in each of the diastole images. The method also includes calculating, by one or more computing devices, a volume of the heart in each of the systoles image based on the orientation of the heart in the respective systole image and the segmentation of the heart in the respective systole image, and a volume of the heart in each of the diastole images based at least on the orientation of the heart in the respective diastole image and the segmentation of the heart in the respective diastole image. The method also includes determining, by one or more computing devices, the heart parameter based at least on the volume of the heart in each systole image and the volume of the heart in each diastole image, and determining, by one or more computing devices, a confidence score of the heart parameter. The method also includes displaying, by one or more computing devices, the heart parameter and the confidence score.

The series of images can cover six heart cycles, and the method can include identifying six systole images and six diastole images. The method can include generating a wall trace of the heart including a deformable spline connected by a plurality of nodes, and displaying the wall trace of the heart in at least one of the systole images and the diastole images. The method can include receiving a user adjustment of at least one node to modify the wall trace. The method can include modifying the wall trace of the heart in one or more other images, based on the user adjustment. The heart parameter can include ejection fraction.

In accordance with the disclosed subject matter, one or more computer-readable non-transitory storage media embodying software are provided. The software is operable when executed to receive a series of two-dimensional images of a heart, the series covering at least one heart cycle, and identify a first systole image from the series of images associated with systole of the heart and a first diastole image from the series of images associated with diastole of the heart. The software is operable when executed to calculate an orientation of the heart in the first systole image and an orientation of the heart in the first diastole image, and calculate a segmentation of the heart in the first systole image and a segmentation of the heart in the first diastole image. The software is operable when executed to calculate a volume of the heart in the first systole image based on the orientation of the heart in the first systole image and the segmentation of the heart in the first systole image, and a volume of the heart in the first diastole image based at least on the orientation of the heart in the first diastole image and the segmentation of the heart in the first diastole image. The software is operable when executed to determine the heart parameter based at least on the volume of the heart in the first systole image and the volume of the heart in the first diastole image, and determine a confidence score of the heart parameter. The software is operable when executed to display the heart parameter and the confidence score.

In accordance with the disclosed subject matter, a system including one or more processors; and a memory coupled to the processors including instructions executable by the processors are provided. The processors are operable when executing the instructions to receive a series of two-dimensional images of a heart, the series covering at least one heart cycle, and identify a first systole image from the series of images associated with systole of the heart and a first diastole image from the series of images associated with diastole of the heart. The processors are operable when executing the instructions to calculate an orientation of the heart in the first systole image and an orientation of the heart in the first diastole image, and calculate a segmentation of the heart in the first systole image and a segmentation of the heart in the first diastole image. The processors are operable when executing the instructions to calculate a volume of the heart in the first systole image based on the orientation of the heart in the first systole image and the segmentation of the heart in the first systole image, and a volume of the heart in the first diastole image based at least on the orientation of the heart in the first diastole image and the segmentation of the heart in the first diastole image. The processors are operable when executing the instructions to determine the heart parameter based at least on the volume of the heart in the first systole image and the volume of the heart in the first diastole image, and determine a confidence score of the heart parameter. The processors are operable when executing the instructions to display the heart parameter and the confidence score.

### DRAWINGS

FIG. 1 shows a hierarchy of medical image records that can be compressed and stored in accordance with the disclosed subject matter.

FIG. 2 shows an architecture of a system for calculating heart parameters, in accordance with the disclosed subject matter.

FIG. 3 illustrates medical image records, in accordance with the disclosed subject matter.

FIG. 4 illustrates medical image records with a 2D segmentation model applied, in accordance with the disclosed subject matter.

FIG. 5 shows a plot of an area trace, in accordance with the disclosed subject matter.

FIG. 6 illustrates a medical image record including an orientation and a segmentation, in accordance with the disclosed subject matter.

FIG. 7 shows a model architecture, in accordance with the disclosed subject matter.

FIGS. 8A and 8B illustrate medical image records including wall traces, in accordance with the disclosed subject matter.

FIG. 9 illustrates a medical image record including a flexible-deformable spline object, in accordance with the disclosed subject matter.

FIG. 10 illustrates a flow chart of a method for calculating heart parameters, in accordance with the disclosed subject matter.

### DETAILED DESCRIPTION

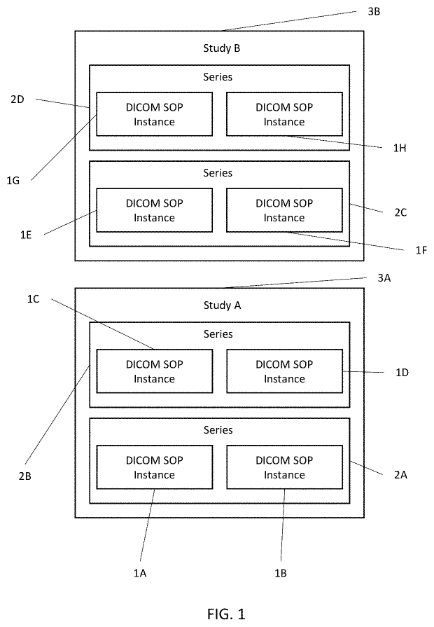

Reference will now be made in detail to various exemplary embodiments of the disclosed subject matter, exemplary embodiments of which are illustrated in the accompanying figures. For purpose of illustration and not limitation, the methods and systems are described herein with respect to determining parameters of a heart (human or animal), however, the methods and systems described herein can be used for determining parameters of any organ having varying volumes over time, for example, a bladder. As used in the description and the appended claims, the singular forms, such as “a,” “an,” “the,” and singular nouns, are intended to include the plural forms as well, unless the context clearly indicates otherwise. Accordingly, as used herein, the term image can be a medical image record and can refer to one medical image record, or a plurality of medical image records. For example, and with reference to FIG. 1 for purpose of illustration and not limitation, as referred to herein a medical image record, which can include a single Digital Imaging and Communications in Medicine (“DICOM”) Service-Object Pair (“SOP”) Instance (also referred to as “DICOM Instance” and “DICOM image”) 1 (e.g., 1A-1H), one or more DICOM SOP Instances 1 (e.g., 1A-1H) in one or more Series 2 (e.g., 2A-D), one or more Series 2 (e.g., 2A-D) in one or more Studies 3 (e.g., 3A, 3B), and one or more Studies 3 (e.g., 3A, 3B). Additionally or alternatively, the term image can include an ultrasound image. The methods and systems described herein can be used with medical image records stored on PACS, however, a variety of records are suitable for the present disclosure and records can be stored in any system, for example a Vendor Neutral Archive (“VNA”). The disclosed systems and methods can be performed in an automated fashion (i.e., no user input once the method is initiated) or in a semi-automated fashion (i.e., with some user input once the method is initiated).

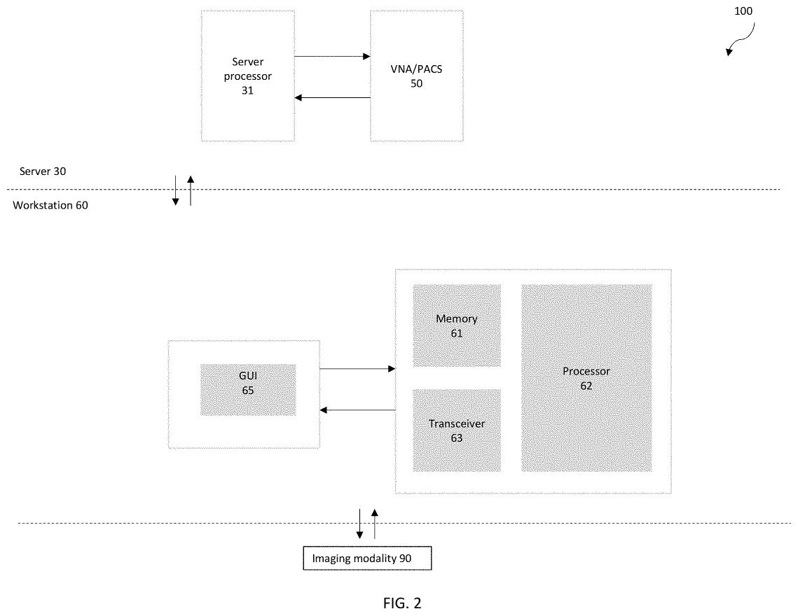

Referring to FIG. 2 for purpose of illustration and not limitation, the disclosed system 100 can be configured to calculate a heart parameter. The system 100 can include one or more computing devices defining a server 30, a user workstation 60, and an imaging modality 90. The user workstation 60 can be coupled to the server 30 by a network. The network, for example, can be a Local Area Network (“LAN”), a Wireless LAN (“WLAN”), a virtual private network (“VPN”), any other network that allows for any radio frequency or wireless type connection, or combinations thereof. For example, other radio frequency or wireless connections can include, but are not limited to, one or more network access technologies, such as Global System for Mobile communication (“GSM”), Universal Mobile Telecommunications System (“UMTS”), General Packet Radio Services (“GPRS”), Enhanced Data GSM Environment (“EDGE”), Third Generation Partnership Project (“3 GPP”) Technology, including Long Term Evolution (“LTE”), LTE-Advanced, 3G technology, Internet of Things (“JOT”), fifth generation (“5G”), or new radio (“NR”) technology. Other examples can include Wideband Code Division Multiple Access (“WCDMA”), Bluetooth, IEEE 802.11b/g/n, or any other 802.11 protocol, or any other wired or wireless connection.

Workstation 60 can take the form of any known client device. For example, workstation 60 can be a computer, such as a laptop or desktop computer, a personal data or digital assistant (“PDA”), or any other user equipment or tablet, such as a mobile device or mobile portable media player, or combinations thereof. Server 30 can be a service point which provides processing, database, and communication facilities. For example, the server 30 can include dedicated rack-mounted servers, desktop computers, laptop computers, set top boxes, integrated devices combining various features, such as two or more features of the foregoing devices, or the like. Server 30 can vary widely in configuration or capabilities, but can include one or more processors, memory, and/or transceivers. Server 30 can also include one or more mass storage devices, one or more power supplies, one or more wired or wireless network interfaces, one or more input/output interfaces, and/or one or more operating systems. Server 30 can include additional data storage such as VNA/PACS 50, remote PACS, VNA, or other vendor PACS/VNA.

The Workstation 60 can communicate with imaging modality 90 either directly (e.g., through a hard wired connection) or remotely (e.g., through a network described above) via a PACS. The imaging modality 90 can include an ultrasound imaging device, such as an ultrasound machine or ultrasound system that transmits the ultrasound signals into a body (e.g., a patient), receives reflections from the body based on the ultrasound signals, and generates ultrasound images from the received reflections. Although described with respect to an ultrasound imaging device, imaging modality 90 can include any medical imaging modality, including, for example, x-ray (or x-ray's digital counterparts: computed radiography (“CR”) and digital radiography (“DR”)), mammogram, tomosynthesis, computerized tomography (“CT”), magnetic resonance image (“MRI”), and positron emission tomography (“PET”). Additionally or alternatively, the imaging modality 90 can include one or more sensors for generating a physiological signal from a patient, such as electrocardiogram (“EKG”), respiratory signal, or other similar sensor systems.

A user can be any person authorized to access workstation 60 and/or server 30, including a health professional, medical technician, researcher, or patient. In some embodiments a user authorized to use the workstation 60 and/or communicate with the server 30 can have a username and/or password that can be used to login or access workstation 60 and/or server 30. In accordance with the disclosed subject matter, one or more users can operate one or more of the disclosed systems (or portions thereof) and can implement one or more of the disclosed methods (or portions thereof).

Workstation 60 can include GUI 65, memory 61, processor 62, and transceiver 63. Medical image records 71 (e.g., 71A, 71B) received by workstation 60 can be processed using one or more processors 62. Processor 62 can be any hardware or software used to execute computer program instructions. These computer program instructions can be provided to a processor of a general purpose computer to alter its function to a special purpose computer, application-specific integrated circuit (“ASIC”), or other programmable digital data processing apparatus, such that the instructions, which execute via the processor of the workstation 60 or other programmable data processing apparatus, implement the functions/acts specified in the block diagrams or operational block or blocks, thereby transforming their functionality in accordance with embodiments herein. The processor 62 can be a portable embedded micro-controller or micro-computer. For example, processor 62 can be embodied by any computational or data processing device, such as a central processing unit (“CPU”), digital signal processor (“DSP”), ASIC, programmable logic devices (“PLDs”), field programmable gate arrays (“FPGAs”), digitally enhanced circuits, or comparable device or a combination thereof. The processor 62 can be implemented as a single controller, or a plurality of controllers or processors. The processor 62 can implement one or more of the methods disclosed herein.

Workstation 60 can send and receive medical image records 71 (e.g., 71A, 71B) from server 30 using transceiver 63. Transceiver 63 can, independently, be a transmitter, a receiver, or both a transmitter and a receiver, or a unit or device that can be configured both for transmission and reception. In other words, transceiver 63 can include any hardware or software that allows workstation 60 to communicate with server 30. Transceiver 63 can be either a wired or a wireless transceiver. When wireless, the transceiver 63 can be implemented as a remote radio head which is not located in the device itself, but in a mast. While FIG. 2 only illustrates a single transceiver 63, workstation 60 can include one or more transceivers 63. Memory 61 can be a non-volatile storage medium or any other suitable storage device, such as a non-transitory computer-readable medium or storage medium. For example, memory 61 can be a random-access memory (“RAM”), read-only memory (“ROM”), hard disk drive (“HDD”), erasable programmable read-only memory (“EPROM”), electrically erasable programmable read-only memory (“EEPROM”), flash memory or other solid-state memory technology. Memory 61 can also be a compact disc read-only optical memory (“CD-ROM”), digital versatile disc (“DVD”), any other optical storage, magnetic cassettes, magnetic tape, magnetic disk storage or other magnetic storage devices, or any other physical or material medium which can be used to tangibly store the desired information or data or instructions and which can be accessed by a computer or processor. Memory 61 can be either removable or non-removable.

Server 30 can include a server processor 31 and VNA/PACS 50. The server processor 31 can be any hardware or software used to execute computer program instructions. These computer program instructions can be provided to a processor of a general purpose computer to alter its function to a special purpose, a special purpose computer, ASIC, or other programmable digital data processing apparatus, such that the instructions, which execute via the processor of the client station or other programmable data processing apparatus, implement the functions/acts specified in the block diagrams or operational block or blocks, thereby transforming their functionality in accordance with embodiments herein. In accordance with the disclosed subject matter, the server processor 31 can be a portable embedded micro-controller or micro-computer. For example, server processor 31 can be embodied by any computational or data processing device, such as a CPU, DSP, ASIC, PLDs, FPGAs, digitally enhanced circuits, or comparable device or a combination thereof. The server processor 31 can be implemented as a single controller, or a plurality of controllers or processors.

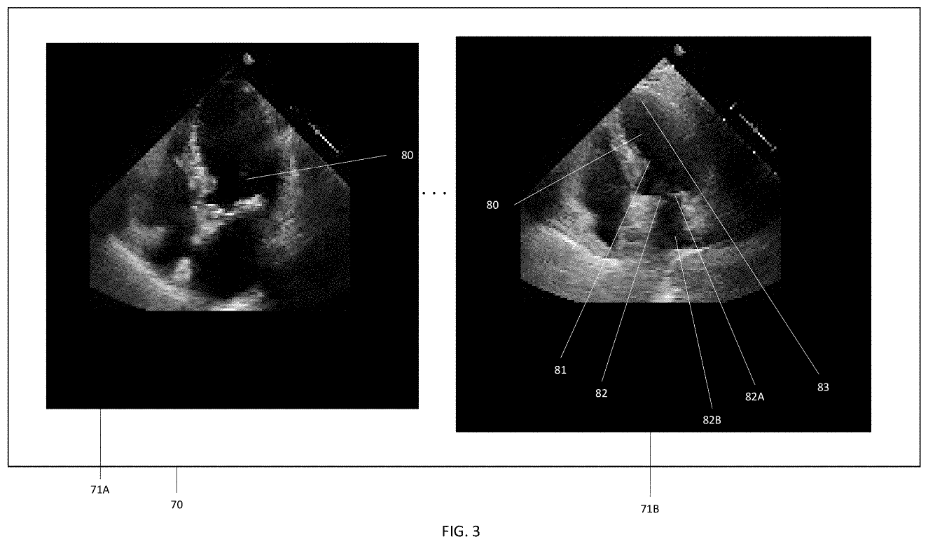

As shown in FIG. 3, images can be a series 70 of two-dimensional images 71 (only images 71A and 71B are shown), for example, images can be a series of ultrasound images covering at least one heart cycle, for example, between one and ten heart cycles. The series 70 of two-dimensional images 71 (e.g., 71A and 71B) can be received directly from imaging device 90. Additionally, or alternatively, the series 70 can be a Series 2 and the two-dimensional images 71 (e.g., 71A, 71B) can be a plurality of DICOM SOP Instances 1. For example, images 71A and 71B are ultrasound images of a mouse heart 80 (although described with respect to a mouse heart, the systems and methods disclosed herein can be used with images of other animal hearts, including images of human hearts) at different points in the cardiac cycle. The images 71 (e.g., 71A, 71B) can be B-mode (bright mode; also sometimes referred to as 2D mode) ultrasound images which can use an array (e.g., a linear or phased array) of transducers to scan a plane and generate an image that can be viewed as a two-dimensional image on a screen, such as GUI 65. The transducer can also be a matrix array or curved linear array transducer. As an example, image 71A can show heart 80 during diastole and image 71B can show heart 80 during systole. The heart can include a left ventricle 81, which can include a base 82 (which corresponds to the location in the left ventricle 81 where the left ventricle 81 connects to the aorta 82B via the aortic valve 82A) and an apex 83. Although the disclosed subject matter is described with respect to B-mode ultrasound images, the disclosed subject matter can also be applied to M-mode (motion mode) images.

In operation, system 100 can be used to detect a heart parameter, such as ejection fraction, of the heart 80 depicted in the images 71 (e.g., 71A, 71B) of series 70. The system 100 can automate the process of detecting the heart parameters, which can remove the element of human subjectivity (which can remove errors) and can facilitate the rapid calculation of the parameter (which reduces the time required to obtain results).

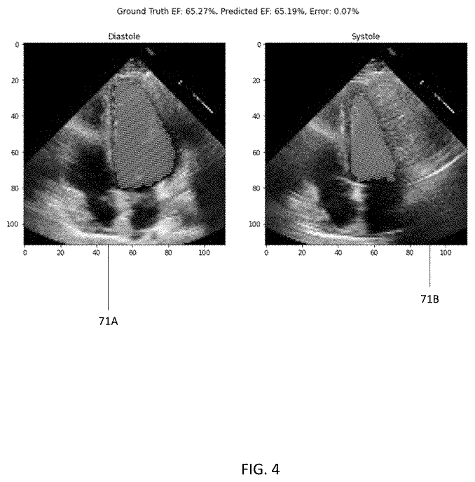

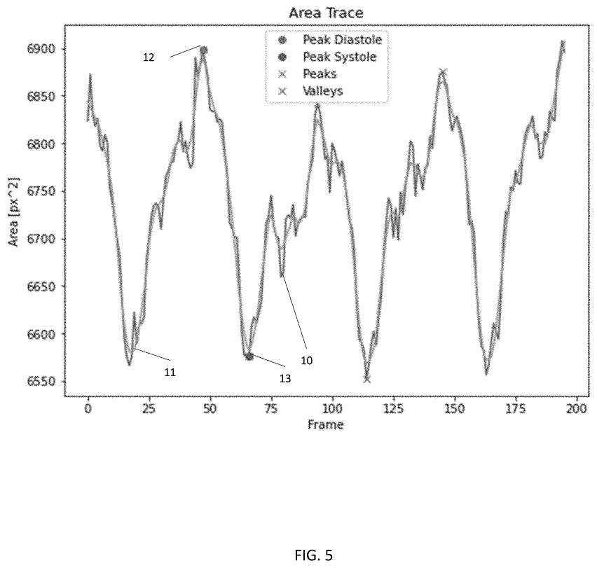

The series 70 of images 71 (e.g., 71A, 71B) can be received by system 100 from imaging modality 90 in real time. The system 100 can identify the image 71 (e.g., 71A, 71B) associated with systole and diastole, respectively. For example, systole and diastole can be determined directly from the images 71 (e.g., 71A, 71B) through computation of the area of the left ventricle 81 in each image 71 (e.g., 71A, 71B). Systole can be the image 71 (e.g., 71B) (or images where several cycles are provided) associated with a minimum area and diastole can be the image 71 (e.g., 71A) (or images where several cycles are provided) associated with a maximum area. The area can be calculated as a summation of the pixels within the segmented region of the left ventricle 81. A model can be trained to perform real-time identification and tracking of the left ventricle 81 in each image 71 (e.g., 71A, 71B) of the series 70. For example, the system 100 can use 2D segmentation model to generate the segmented region, for example, as shown in images 71A and 71B in FIG. 4. One example of a segmented model for identifying and segmenting objects includes a convolutional neural network. System 100 can apply post processing and filtering of the area to remove jitter and artifacts. For example, a moving average window, such as a finite impulse response (FIR) filter can be used. System 100 can apply a peak detection algorithm to identify peaks and valleys. For example, a threshold method can determine when the signal crosses a threshold and reaches a minimum or maximum. System 100 can plot the area of each left ventricle 81, for example, as shown in FIG. 5.

FIG. 5 shows a plot of the area (y-axis) of the ventricle 81 in each frame (x-axis) and illustrates at least three full heart cycles. It is understood that the volume of the heart is related to the area of the heart. For example, as the area of a circle is area=π*r*r, the volume of a sphere with the same radius as the circle is volume=4/3*r*area. The plot includes trace 10 associated with the volume of the LV, trace 11 which is a smoothed version of trace 10, point 12, which is a local maximum (and therefore identifies an image 71 (e.g., 71A, 71B) (frame) associated with diastole), and a point 13, which is a local minimum (and therefore identifies an image 71 (e.g., 71A, 71B) (frame) associated with systole). Additionally, or alternatively, diastole and systole can be identified from a received ECG signal if it is available. The model used can be the final LV segmentation model or a simpler version designed to execute extremely quickly. As the accuracy of the segmentation is not critical to determination of the maxima and minima representing diastole and systole, it can be less accurate and thus more efficient to run in real-time.

In another embodiment, the model can be trained to identify diastole and systole directly from a sequence of images, based on image features. For example using a recurrent neural network (“RNN”), a sequence of images is used as input, and from that sequence the frames which correspond to diastole and systole can be marked.

In accordance with the disclosed subject matter, the system 100 can determine a quality metric of the images in the series of two-dimensional images. The system can confirm that the quality metric is above a threshold. For example, if the quality metric is above the threshold, the system 100 can proceed to calculate the volume; if the quality metric is below the threshold, the images will not be used for determining the volume.

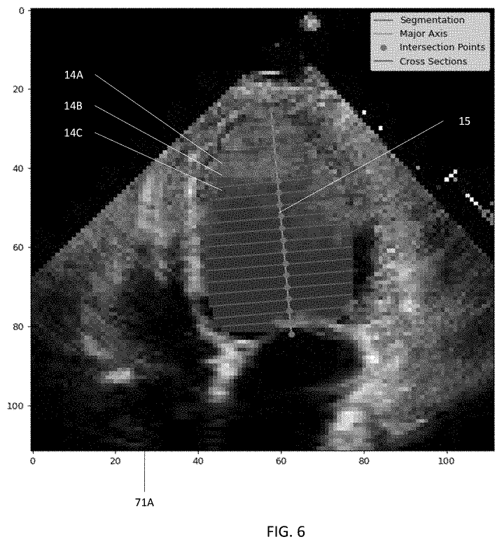

The volume calculation for the left ventricle 81 for each of the images 71 (e.g., 71A, 71B) identified as diastole and systole can be a two-step process including (1) segmentation of the frame; and (2) computation of the orientation. For example, and as shown in FIG. 6, the left ventricle 81 of image 71A has been segmented into a plurality of segmentations 14 (e.g., 14A, 14B, 14C) and the major axis 15 has been plotted, which defines the orientation of the left ventricle 81. These two features can be important because if the orientation is not correct, then the 2D region cannot accurately represent the correct volume, for example, because the calculation, as set forth below, would be rotated around the wrong axis.

To calculate the segmentation of the frame and the orientation, the system 100 can identify the interior (endocardial) and heart wall boundary. This information can be used to obtain the measurements needed to calculate cardiac function metrics. The system 100 can perform the calculation using a model trained with deep learning. The model can be created using (1) an abundance of labeled input data; (2) a suitable deep learning model; and (3) successful training of the model parameters.

For example, the model can be trained using 2,000 data sets, or another amount, for example, 1,000 data sets or 5,000 data sets, collected in the parasternal long-axis view, and with the inner wall boundaries fully traced over a number of cycles. The acquisition frame rate, which can depend on the transducer and imaging settings used, can vary from 20 to 1,000 frames per second (fps). Accordingly, 30 to 100 individual frames can be traced for each cine loop. As clear to one skilled in the art, more correctly-labeled training data generally results in better AI models. A collection of over 150,000 unique images can be used for training. Training augmentation can include horizontal flip, noise, rotations, sheer transformations, contrast, brightness, and deformable image warp. In some embodiments, Generative Adversarial Networks (“GANS”) can be used to generate additional training data. A model using data organized as 2D or 3D sets can be used, however, a 2D model can provide simpler training. For example, a 3D model taking as input a series of images in sequence through the heart cycle, or a sequence of diastole/systole frames can be used. A human evaluation data set can include approximal 10,000 images at 112×112, or other resolutions, for example, 128×128 or 256×256 pixels with manually segmented LV regions. As one skilled in the art would appreciate, different configurations can balance accuracy with inference (execution) time for the model. In a real-time situation a smaller image can be beneficial to maintain processing speed at the cost of some accuracy.

A U-Net model with an input output size of 128×128 can be trained on a segmentation map of the inner wall region. Other models can be used, including DeepLab, EfficientDet, or MobileNet frameworks, or other suitable models. The model architecture can be designed new or can be a modified version of the aforementioned models. Although one skilled in the art will recognize that the number of parameters in the models can vary, the more parameters, typically, the slower the processing time at inference. However, usage of external AI processors, higher end CPUs, embedded and discrete GPUs can improve processing efficiency.

In one example, an additional model configured to identify orientation of the heart can identify the apex and base points of the heart, the two outflow points, or a slope/intercept pair. The model can output two or more data points (e.g., a set of xy data pairs) or directly the slope and intercept point of the heart orientation. Additionally, or alternately, the model used to compute the LV segmentation can also directly generate this information. For example, the segmentation model can generate as a separate output a set of xy data pairs corresponding to the apex and outflow points or the slope and intercept of the orientation line. Alternatively, the model as a separate output channel, can encode the points of the apex and outflow as regions which, using post processing, can identify these positions.

Training can be performed, for example on an NVIDIA VT100 GPU and can use a TensorFlow/Keras-based training framework. As one skilled in the art would appreciate, other deep learning enabled processors can be used for training. As well, other model frameworks such as PyTorch can be used for training. Other training hardware and other training/model frameworks will become available and are interchangeable.

Deep learning models can use separate models to train for identification of segmentation and orientation, respectively, or a combined model trained to identify both features with separate outputs for each data type. Training models separately allows each model to be trained and tested independently. As an example, the models can run in parallel, which can improve efficiency. Additionally or alternatively, models used to determine the diastole and systole frames can be the same as the LV segmentation model, which is a simple solution, or different, which can enable optimizations to the diastole/systole detection model.

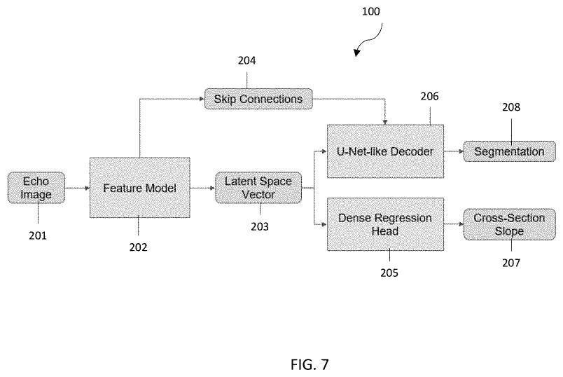

As an example, the models can be combined as shown in the model architecture 200 of FIG. 7. The system can have a single input (e.g., echo image 201) and two outputs (e.g., cross-section slope 207, representing the orientation, and segmentation 208). Alternatively, the model as a separate output channel, can encode the points of the apex and outflow as regions which, using post processing, can identify these positions. As one skilled in the art would appreciate, U-Net is a class of models that can be trained with a relatively small number of data sets to generate segmentations on medical images with little processing delay. The feature model 202 can include an encoder that generates a feature vector from the echo image 201 and this is represented as latent space vector 203. For example, the feature vector generated by the feature model 202 belongs to a latent vector space. One example of an encoder of feature model 202 is a convolutional neural network that includes multiple layers that progressively downsample, thus forming the latent space vector 203. The U-net-like decoder 206 can include a corresponding number of convolutional layers that progressively upsample the latent space vector 203 to generate a segmentation 208. To increase execution speed, layers of the feature model 202 can be connected to corresponding layers of the decoder 206 via skip connections 204, rather than having signals propagate through all layers of the feature model 202 and the decoder 206. The dense regression head 205 can include a network to generate a cross-section slope 207 from the feature vector (e.g., the latent space vector 203). One example of dense regression head 205 includes multiple layers of convolutional layers that are each followed by layers made up of activation functions, such as rectified linear activation functions.

If the model contains more than one output node, it can be trained in a single pass. Alternatively, it can be trained in two separate passes whereby the segmentation output is trained first, at which point the encoding stages parameters are locked, and only the parameters corresponding to the orientation output are trained. Using two separate passes is a common approach with models containing two distinct types of outputs which do not share a similar dimension or shape or type. The training model can be selected based on inference efficiency, accuracy, and implementation simplicity and can be different for different hardware and configurations. Additional models can include sequence networks, RNNS, or networks consisting of embedded LSTM, GRU, or other recurrent layers. The models can be beneficial in that they can utilize prior frame information rather than the instantaneous snapshot of the current frame. Other solutions can utilize 2D models where the input channels are not just the single input frame but can include a number of previous frames. As an example, instead of providing the previous frame, the previous segmentation region can be provided. Additional information can be layered as additional channels to the input data object.

Using the segmentation and the orientation, system 100 can calculate the volume using calculus or other approximations such as a “method of disks” or “Simpson's method,” where the volume is the summation of a number of disks using the equation shown below:

$$V = \frac{π}{4} h {\sum }_{i = 0}^{n} {d}^{2} \qquad ( 2 )$$

where d is a diameter of each segmentation and h is the height of the left ventricle 81 along its orientation (e.g., the major axis).

Multiple pairs of systole and diastole in sequence can be used to improve overall accuracy of the calculation. For example, in a sequence of systole-diastole “S D S D S D S” six separate ejection fractions can be calculated and can improve the overall accuracy of the calculation. This approach can also give a measure of accuracy (also referred to herein as a confidence score) to the user by calculation of metrics such as standard deviation or variance. The ejection fraction value, or other metrics, can be presented directly to the user in a real time scenario. For example, the confidence score can help inform the user if the detected value is accurate. For instance, a standard deviation measures how much the measurements per each cycle vary. A large variance can indicate that the patient heart cycle is changing too rapidly and thus the measurements are inaccurate. The metrics can be based on the calculated EF value or other measures such as the heart volume, area, or position. For example, if the heart is consistently in the same position, as measured by an intersection-over-union calculation of the diastolic and systolic segmentation regions, then the confidence that the calculations are accurate increases. The confidence score can be displayed as a direct measure of the variance or interpreted and displayed as a relative measure; for example “high quality”, “medium quality”, “poor quality”. In some embodiments an additional model, trained to classify good heart views can be trained and used to provide additional metrics on the heart view used and its suitability for EF calculations.

As used herein, “real-time” data acquisition does not need to be 100% in synchronization with image acquisition. For example, acquisition of images can occur at about 30 fps. Although complete ejection fraction calculation can be slightly delayed, a user can still be provided with relevant information. For example, the ejection fraction value does not change dramatically over a short period of time. Indeed, ejection fraction as a measurement requires information from a full heart cycle (volume at diastole and volume at systole). Additionally or alternatively, a sequence of several systole frames can be batched together before ejection fraction is calculated. Thus, the value for ejection fraction can be delayed by one or more heart cycles. This delay can allow a more complex AI calculation to run than might be able to run at the 30 fps rate of image acquisition. Accordingly, a value delayed by for example, up to 5 seconds (for example 1 second) is considered “real time” as used herein. However, it is further noted that not all frames are required to be used for the volume calculation. Rather, one or more frames associated with systole or diastole can be used. In some embodiments initial results can be displayed immediately after 1 heart cycle and then updated as more heart cycles are acquired and the calculations repeated. For example, as more heart cycles are acquired, an average EF of the previous heart cycles can be displayed. Additionally, or alternatively, out of a set of heart cycles, one or more heart cycles can provide incorrect calculations because of patient motion, or temporary incorrect positioning of the probe. The displayed cardiac parameters can exclude these cycles from the final average improving the accuracy of the calculation.

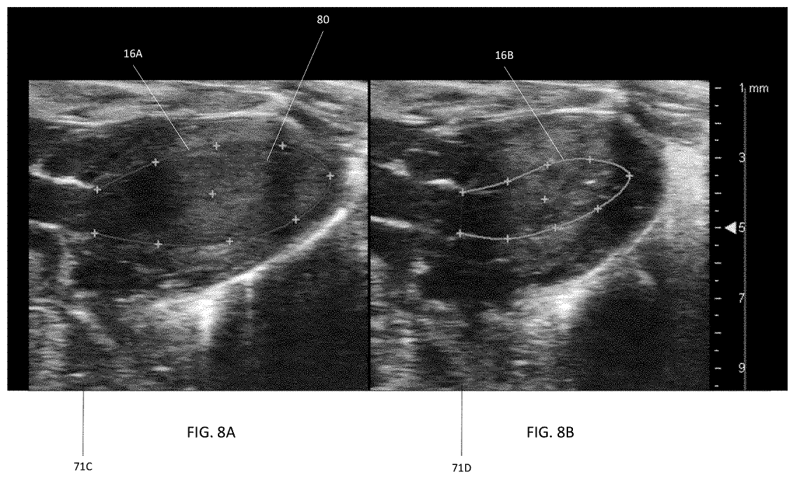

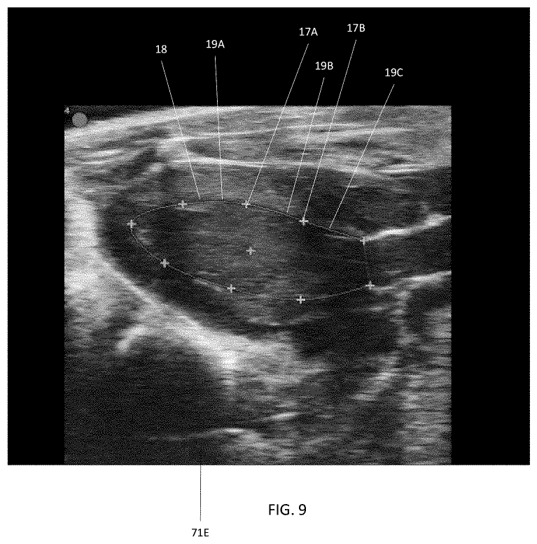

Referring to FIGS. 8A, 8B, and 9, for purpose of illustration and not limitation, a segmentation or heart wall trace 16 (e.g., 16A, 16B) can be drawn on one or more systole and diastole images in real time. This information can be presented to the user and can provide the user a confidence that the traces appear in the correct area. In accordance with the disclosed subject matter, the user can verify the calculation in a review setting. For example, when acquisition (imaging and initial ejection fraction analysis) has been completed, the user can be presented with the recent results of the previous acquisition, which can be based on some amount of time (previous few seconds or previous few minutes) of data before the pause. The data can be annotated with a simplified wall trace 16 (e.g., 16A, 16B) data on each diastole and systole frame, for example, as shown in FIG. 8A on image 71C, which shows a mouse heart in diastole, and FIG. 8B on image 71D, which shows a mouse heart in systole. As shown in FIG. 9, the trace 16 can be reduced to a flexible-deformable spline object 18, such as a Bezier spline. For example, there can be 9 control points 17 (e.g., 17A, 17B) and splines 19 (e.g., 19A-19C) in the deformable spline object 18. The number of control points 17 (e.g., 17A, 17B) can be reduced or increased as desired, e.g., by a user selection. Adjusting any control point 17 (e.g., 17A, 17B) can move the connected splines 19 (e.g., 19A-19C). For example, moving control point 17A can adjust the position of splines 19A and 19B; while moving control point 17B can adjust the positions of splines 19B and 19C. Additionally or alternatively, the entire deformable spline object 18 can be resized, rotated, or translated to adjust its position as required. This ability can provide a simple, fast way to change the shape of the spline object 18.

Once a user has adjusted the shape of any particular spline object 18, the change can be propagated to neighboring images 71 (e.g., 71A-71E). For example, if the user adjusts the spline object 18 for image 71E, which depicts systole, the spline objects 18 for neighboring images 71 (e.g., 71A-71E) depicting systole can be adjusted using frame adaptation methods. It can be understood that within a short period of time, over a range of several heart cycles, all of the systole (or diastole) frames are similar to other frames depicting systole (or diastole). The similarities between frames can be estimated. If they are similar, then the results of one frame can be translated to the other frames using methods such as optical flow. The frame the user adjusted can be warped to neighboring systole frames using optical flow, as it can be understood the other frames require similar adjustments as applied by the user to the initial frame. In accordance with the disclosed subject matter, a condition can be added that once a frame is manually adjusted it is not adjusted in future propagated (automatic) adjustments.

In accordance with the disclosed subject matter, an algorithm configured for real-time computation of ejection fraction (for example, an algorithm that can present ejection fraction while a user is imaging a heart) can be simpler and faster than an algorithm configured for post-processing computation of ejection fraction. For example, during imaging a real-time computation of ejection fraction can be presented to the user. Upon pausing acquisition of images the system 100 can run a more complex algorithm and provide a computation of ejection fraction based on a more complex algorithm. Accordingly, the system 100 can generate heart parameters, such as ejection fraction, when traditional systems that merely post process images are too slow to be useful. Moreover, the system 100 can generate more accurate heart parameters than traditional systems and display indications of that accuracy via a confidence score, as described above, thus reducing operator-induced errors.

Although ejection fraction is calculated based on the volume as systole and diastole, area and volume calculations over an entire heart cycle can be useful. Accordingly, trace objects 18 can be generated for all frames (including systole and diastole). This generation can be done by repeating the processes described above, and can include the following workflow: (1) select a region of a data set to process (for example part of a heart cycle, all of a heart cycle, or multiple heart cycles); (2) performed segmentation on each frame; (3) perform intra-frame comparisons to remove anomalous inference results; (4) compute edges of each frame; (5) identify apex and outflow points; and (6) generate smooth splines from edge map. Additionally or alternatively, optical flow can be used to generate frames between the already computed diastole-systole frame pairs. This process can incorporate changes made by the user to the diastole and systole spline objects 18.

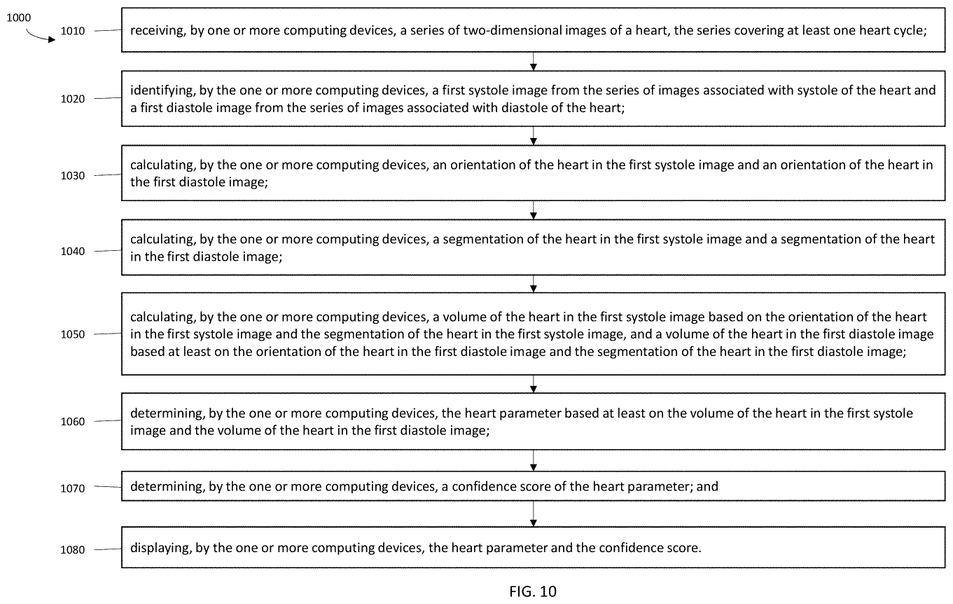

FIG. 10 illustrates an example method 1000 for calculating a heart parameter. The method 1000 can be performed by processing logic that can include hardware (e.g., circuitry, dedicated logic, etc.), software (such as is run on a general-purpose computer system or a dedicated machine), firmware (e.g., software programmed into a read-only memory), or combinations thereof. In some embodiments, the method 1000 is performed by an ultrasound machine.

The method 1000 can begin at step 1010, where the method includes receiving, by one or more computing devices, a series of two-dimensional images of a heart, the series covering at least one heart cycle. At step 1020, the method includes identifying, by one or more computing devices, a first systole image from the series of images associated with systole of the heart and a first diastole image from the series of images associated with diastole of the heart. At step 1030, the method includes calculating, by one or more computing devices, an orientation of the heart in the first systole image and an orientation of the heart in the first diastole image. At step 1040, the method includes calculating, by one or more computing devices, a segmentation of the heart in the first systole image and a segmentation of the heart in the first diastole image. At step 1050, the method includes calculating, by one or more computing devices, a volume of the heart in the first systole image based on the orientation of the heart in the first systole image and the segmentation of the heart in the first systole image, and a volume of the heart in the first diastole image based at least on the orientation of the heart in the first diastole image and the segmentation of the heart in the first diastole image. At step 1060, the method includes determining, by one or more computing devices, the heart parameter based at least on the volume of the heart in the first systole image and the volume of the heart in the first diastole image. At step 1070, the method includes determining, by one or more computing devices, a confidence score of the heart parameter. At step 1080, the method includes displaying, by one or more computing devices, the heart parameter and the confidence score.

In accordance with the disclosed subject matter, the method can repeat one or more steps of the method of FIG. 10, where appropriate. Although this disclosure describes and illustrates particular steps of the method of FIG. 10 as occurring in a particular order, this disclosure contemplates any suitable steps of the method of FIG. 10 occurring in any suitable order. Moreover, although this disclosure describes and illustrates an example method for calculating a heart parameter including the particular steps of the method of FIG. 10, this disclosure contemplates any suitable method for calculating a heart parameter including any suitable steps, which can include all, some, or none of the steps of the method of FIG. 10, where appropriate. Furthermore, although this disclosure describes and illustrates particular components, devices, or systems carrying out particular steps of the method of FIG. 10, this disclosure contemplates any suitable combination of any suitable components, devices, or systems carrying out any suitable steps of the method of FIG. 10.

As described above in connection with certain embodiments, certain components, e.g., server 30 and workstation 60, can include a computer or computers, processor, network, mobile device, cluster, or other hardware to perform various functions. Moreover, certain elements of the disclosed subject matter can be embodied in computer readable code which can be stored on computer readable media (e.g., one or more storage memories) and which when executed can cause a processor to perform certain functions described herein. In these embodiments, the computer and/or other hardware play a significant role in permitting the system and method for calculating a heart parameter. For example, the presence of the computers, processors, memory, storage, and networking hardware provides the ability to calculate a heart parameter in a more efficient manner. Moreover, storing and saving the digital records cannot be accomplished with pen or paper, as such information is received over a network in electronic form.

The subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium also can be, or may be included in, one or more separate physical components or media (e.g., multiple CDs, disks, or other storage devices).

The term processor encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA or an ASIC. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program can, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA or an ASIC.

Processors suitable for the execution of a computer program can include, by way of example and not by way of limitation, both general and special purpose microprocessors. Devices suitable for storing computer program instructions and data can include all forms of non-volatile memory, media and memory devices, including by way of example but not by way of limitation, semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Additionally, as described above in connection with certain embodiments, certain components can communicate with certain other components, for example via a network, e.g., a local area network or the internet. To the extent not expressly stated above, the disclosed subject matter is intended to encompass both sides of each transaction, including transmitting and receiving. One of ordinary skill in the art will readily understand that with regard to the features described above, if one component transmits, sends, or otherwise makes available to another component, the other component will receive or acquire, whether expressly stated or not.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1) A method for calculating a heart parameter, comprising:
receiving, by one or more computing devices, a series of two-dimensional images of a heart, the series covering at least one heart cycle;
identifying, by the one or more computing devices, a first systole image from the series of images associated with systole of the heart and a first diastole image from the series of images associated with diastole of the heart;
calculating, by the one or more computing devices, an orientation of the heart in the first systole image and an orientation of the heart in the first diastole image;
calculating, by the one or more computing devices, a segmentation of the heart in the first systole image and a segmentation of the heart in the first diastole image;
calculating, by the one or more computing devices, a volume of the heart in the first systole image based on the orientation of the heart in the first systole image and the segmentation of the heart in the first systole image, and a volume of the heart in the first diastole image based at least on the orientation of the heart in the first diastole image and the segmentation of the heart in the first diastole image;
determining, by the one or more computing devices, the heart parameter based at least on the volume of the heart in the first systole image and the volume of the heart in the first diastole image;
determining, by the one or more computing devices, a confidence score of the heart parameter; and
displaying, by the one or more computing devices, the heart parameter and the confidence score.

2) The method of claim 1, further comprising determining areas for the heart including an area for the heart for each image in the series of images, wherein the identifying the first systole image is based on identifying a smallest area among the areas, the smallest area representing a smallest heart volume.

3) The method of claim 1, further comprising determining areas for the heart including an area for the heart for each image in the series of images, wherein identifying the first diastole image is based on identifying a largest area among the areas, the largest area representing a largest heart volume.

4) The method of claim 1, wherein the calculating the orientation of the heart in the first systole image and the orientation of the heart in the first diastole image is based on a deep learning algorithm.

5) The method of claim 1, further comprising identifying a base and an apex of the heart in each of the first systole image and the first diastole image, wherein the calculating the orientation of the heart in the first systole image and the orientation of the heart in the first diastole image is based on the base and the apex in the respective image.

6) The method of claim 1, wherein the calculating the segmentation of the heart in the first systole image and the segmentation of the heart in the first diastole image is based on a deep learning algorithm.

7) The method of claim 1, further comprising determining a border of the heart in each of the first systole image and the first diastole image, wherein the calculating the segmentation of the heart in the first systole image and the segmentation of the heart in the first diastole image is based on the orientation of the heart in the respective image and the border of the heart in the respective image.

8) The method of claim 1, further comprising:
generating a wall trace of the heart including a deformable spline connected by a plurality of nodes; and
displaying the wall trace of the heart in one of the first systole image and the first diastole image.

9) The method of claim 8, further comprising receiving a user adjustment of at least one node to modify the wall trace.

10) The method of claim 9, further comprising modifying the wall trace of the heart in the other of the first systole image and the first diastole image, based on the user adjustment.

11) The method of claim 1, wherein the heart parameter includes an ejection fraction.

12) The method of claim 1, wherein the determining the heart parameter includes determining the heart parameter in real time.

13) The method of claim 1, further comprising determining a quality metric of the images in the series of two-dimensional images; and
confirming that the quality metric is above a threshold.

14) A method for calculating a heart parameter, comprising:
receiving, by one or more computing devices, a series of two-dimensional images of a heart, the series covering a plurality of heart cycles;
identifying, by the one or more computing devices, a plurality of systole images from the series of images, each associated with systole of the heart and a plurality of diastole images from the series of images, each associated with diastole of the heart;
calculating, by the one or more computing devices, an orientation of each heart in each of the systole images and an orientation of the heart in each of the diastole images;
calculating, by the one or more computing devices, a segmentation of the heart in each of the systole images and a segmentation of the heart in each of the diastole images;
calculating, by the one or more computing devices, a volume of the heart in each of the systoles image based on the orientation of the heart in the respective systole image and the segmentation of the heart in the respective systole image, and a volume of the heart in each of the diastole images based at least on the orientation of the heart in the respective diastole image and the segmentation of the heart in the respective diastole image;
determining, by the one or more computing devices, the heart parameter based at least on the volume of the heart in each systole image and the volume of the heart in each diastole image;
determining, by the one or more computing devices, a confidence score of the heart parameter; and
displaying, by the one or more computing devices, the heart parameter and the confidence score.

15) The method of claim 14, wherein the series of images covers six heart cycles, and the method comprises identifying six systole images and six diastole images.

16) The method of claim 14, further comprising:
generating a wall trace of the heart including a deformable spline connected by a plurality of nodes; and
displaying the wall trace of the heart in at least one of the systole images and the diastole images.

17) The method of claim 16, further comprising receiving a user adjustment of at least one node to modify the wall trace.

18) The method of claim 17, further comprising modifying the wall trace of the heart in one or more other images, based on the user adjustment.

19) The method of claim 14, wherein the heart parameter includes an ejection fraction.

20) One or more computer-readable non-transitory media embodying software that is operable when executed to:
receive a series of two-dimensional images of a heart, the series covering at least one heart cycle;
identify a first systole image from the series of images associated with systole of the heart and a first diastole image from the series of images associated with diastole of the heart;
calculate an orientation of the heart in the first systole image and an orientation of the heart in the first diastole image;
calculate a segmentation of the heart in the first systole image and a segmentation of the heart in the first diastole image;
calculate a volume of the heart in the first systole image based on the orientation of the heart in the first systole image and the segmentation of the heart in the first systole image, and a volume of the heart in the first diastole image based at least on the orientation of the heart in the first diastole image and the segmentation of the heart in the first diastole image;
determine the heart parameter based at least on the volume of the heart in the first systole image and the volume of the heart in the first diastole image;
determine a confidence score of the heart parameter; and
display the heart parameter and the confidence score.

21) A system comprising: one or more processors; a memory coupled to the processors comprising instructions executable by the processors, the processors being operable when executing the instructions to:
receive a series of two-dimensional images of a heart, the series covering at least one heart cycle;
identify a first systole image from the series of images associated with systole of the heart and a first diastole image from the series of images associated with diastole of the heart;
calculate an orientation of the heart in the first systole image and an orientation of the heart in the first diastole image;
calculate a segmentation of the heart in the first systole image and a segmentation of the heart in the first diastole image;
calculate a volume of the heart in the first systole image based on the orientation of the heart in the first systole image and the segmentation of the heart in the first systole image, and a volume of the heart in the first diastole image based at least on the orientation of the heart in the first diastole image and the segmentation of the heart in the first diastole image;
determine the heart parameter based at least on the volume of the heart in the first systole image and the volume of the heart in the first diastole image;
determine a confidence score of the heart parameter; and
display the heart parameter and the confidence score.
